(19) United States
(12) Patent Application Publication
(10) Pub. No.: US 2009/0124572 A1
(43) Pub. Date: May 14, 2009

(54) Iron-containing nutritional supplement

(76) Inventor: Deanna Jean Nelson, Raleigh, NC (US)

Correspondence Address: Kent Barta S.C., 2901 Jonathan Cir., Fitchburg, WI, 53711, US

(21) Appl. No.: 11/983,545

(22) Filed: Nov. 9, 2007

(51) Int. Cl. A61K 31/70; A61K 31/295; A61K 31/4415; A61K 31/4406; A61P 3/02; A61K 31/34; A61K 31/525

(52) U.S. Cl. 514/52; 514/502; 514/251; 514/348; 514/474; 514/356

(57) ABSTRACT

The present invention relates to a nutritional supplement, and particularly, to an oral nutritional supplement which contains an iron fortificant comprising a ferric pyrophosphate chelate. The nutritional supplement can also include vitamins, non-ferrous minerals, and other ingredients. The nutritional supplement is useful for providing iron to animals, and is intended to be used, for example, to administer iron to animals and humans, including individuals afflicted with anemia of chronic disease, pregnant women, women anticipating pregnancy, and lactating women. The compositions and methods can also be used to administer iron together with one or more vitamins or non-ferrous minerals to men, women, children or infants, as well as to animals.

### FIELD OF THE INVENTION

The present invention relates to a nutritional supplement, and particularly, to an oral nutritional supplement which contains an iron fortificant comprising a ferric pyrophosphate chelate. The nutritional supplement can also include vitamins, non-ferrous minerals, and other ingredients.

### BACKGROUND OF THE INVENTION

Iron deficiency is the world's most prevalent nutrient deficiency and causes significant economic losses to both individuals and entire countries in the developing world. In humans, a sufficient supply of iron is essential for the functioning of many biological processes, including binding and transport of oxygen, cardiac function, immune function, neurological function, electron transport, gene regulation, and regulation of cell growth and differentiation. The consequences of iron deficiency include, therefore, not only anemia (as measured by hemoglobin status) but also impaired thermoregulation, impaired thyroid function, impaired immune function, impaired mental function, impaired cognitive development, impaired physical performance (including the ability to perform the usual and customary tasks of daily living), complications of pregnancy, increased absorption of lead and cadmium, altered drug metabolism, increased insulin sensitivity, glossitis, angular stomatitis, koilonychia (spoon nails), pica (behaviorial disturbances characterized by abnormal consumption of non-food items), blue sclera, fatigue, and restless leg syndrome.

Iron fortification of foods has effectively alleviated iron deficiency in the general populations of developed countries. This approach, however, may be insufficient to supply the daily iron requirements in vulnerable groups. For example, the dietary intake of iron by infants, children, young women, and the elderly often fails to match physiological requirements, even in developed countries. Likewise, where intestinal iron uptake has been compromised by chronic inflammation or where iron in blood losses exceeds dietary intake, insufficient iron uptake and the resulting iron deficiency may exacerbate the disease state and increase the risk of death. [This condition is frequently observed in patients suffering anemia of chronic disease (ACD), e.g., patients with chronic infections, cancer, autoimmune disorders, inflammatory bowel disease or end-stage renal disease (ESRD).] Finally, local diets in developing countries may limit iron availability, as these diets consist largely of cereal grains and legumes, and tea, coffee, cocoa and certain vegetables containing iron-uptake inhibitors (e.g., phytates and polyphenols, respectively).

Normally, physiological iron deficiencies are corrected by the absorption of 1-3 mg/day of iron from the gastrointestinal tract. Iron salts such as ferrous sulfate are relatively inexpensive oral iron supplements, costing less than $10 per month. Therefore, to counter iron deficiency, the majority of iron-deficient patients take oral iron supplements 2-3 times a day in addition to a number of other essential medications, including therapeutic agents for the disease state and co-morbidities, co-medicaments to retard the progress of the disease (e.g., phosphate binders for ESRD patients), multivitamins, etc. Patients with anemia of chronic disease(s), however, may suffer from depressed iron absorption due to chronic inflammation. In addition, these patients are at increased risk of gastrointestinal toxicity from iron supplement administration including dyspepsia, anorexia and impaired taste, since a significant proportion of these patients suffer from uremic gastritis, drug-induced gastritis, and diabetic gastroparesis. Further, absorption of oral iron may be impaired secondary to co-administration of other medications such as phosphate binders with food (e.g., ion-exchange resins, calcium or lanthanum salts).

Dietary Reference Intakes (DRI) for Iron. The DRI for iron varies with age and gender, ranging from 8 mg iron/person/day for adult men 19-70+ years of age to 18 mg iron/person/day for menstruating women 19-50 years of age. [“Dietary Reference Intakes for Vitamin A, Vitamin K, Arsenic, Boron, Chromium, Copper, Iodine, Iron, Manganese, Molybdenum, Nickel, Silicon, Vanadium, and Zinc,” Food and Nutrition Board, Institute of Medicine, National Academies, 2001. Accessed via http://www.nap.edu.] The DRIs for infants (7-12 months), children, adolescents, and teens are in this range. A DRI has not been set for infants 0-6 months of age. A DRI of 27 mg iron/person/day is indicated for pregnant women; the DRI is reduced to 9-10 mg iron/person/day when breast-feeding post-partum.

The upper limit (UL) for iron established by the Institute of Medicine is 45 mg iron/person/day for adults (of 19 years of age or older) and adolescents (14-18 years) and 40 mg iron/person/day for infants (0-12 months) and children (1-13 years). The UL represents the highest level of daily iron intake that is likely to pose no risk of adverse health effects in almost all individuals. Individuals with hereditary hemochromatosis, liver disease, or iron loading abnormalities are exceptionally sensitive to the effects of iron overload and were not considered in the derivation of a UL for the general population.

Conventional Iron Fortificants. The iron compounds which are used or have been studied as iron fortificants in nutritional supplements include ferrous sulfate, ferrous fumarate, ferrous folate, an iron dextran, ferric oxyhydroxide dextran, a chitosan derivative of iron, an oligosaccharide derivative of iron, ferrous acetyl salicylate, ferrous gluconate, ferrous diphosphate, carbonyl iron, ferric orthophosphate, ferrous glycine sulfate, ferrous chloride, ferrous ammonium citrate, ferric ammonium citrate, ferric ammonium tartrate, ferric phosphate, ferric potassium tartrate, ferric albuminate, ferric cacodylate, ferric hydroxide, ferric pyrophosphate, ferric quinine citrate, ferric valerate, saccharated iron oxide, iron oxide, ferric chloride, ferrous iodide, ferrous nitrate, ferrous glycerophosphate, ferrous formate, an amino acid and iron salt, an iron salt of a protein hydrolysate, ferrous lactate, ferrous tartrate, ferrous succinate, ferrous glutamate, ferrous citrate, ferrous pyrophosphate, ferrous choline isocitrate, ferrous carbonate, an iron-sugar-carboxylate chelate, ferrous sucrate malate, ferrous sucrate citrate, ferrous fructate citrate, ferrous sucrate ascorbate, ferrous fructate ascorbate, sodium iron EDTA (NaFeEDTA), and ferrous bisglycinate chelate.

In general, water-soluble iron(II) compounds have the highest relative bioavailability of the conventional iron sources but frequently cause unacceptable sensory changes after ingestion or deleterious changes in food quality. Ferrous sulfate is the most commonly used, water-soluble iron fortificant and is found in infant formula, bread and pasta, and iron supplements. It can also be added to wheat flour when stored for short periods but may provoke fat oxidation and “off-flavors” in milk, wheat and other cereal flours stored for longer periods. Pestaner et al. have stated, “Ferrous sulfate is the cheapest, most toxic, and most frequently used iron supplement and has an elemental iron content of approximately 20%.” [J. P. Pestaner, K. G. Ishak, F. G. Mullick, J. A. Centeno, Ferrous sulfate toxicity: a review of autopsy findings, Biolog. Trace Element Res 69: 191-198, 1999] Ferrous sulfate is very soluble in water and aqueous solutions, dissolves to provide solutions having a strongly acid pH of about 2, and is described as a corrosive agent on related Material Safety Data Sheets.

A more expensive alternative to ferrous sulfate, NaFeEDTA, offers the advantages that it has equivalent bioavailability and prevents iron binding to iron absorption inhibitors, particularly phytate. Further, it does not catalyze fat oxidation in stored wheat flour. Concerns about renal toxicity of EDTA, however, may deter use of NaFeEDTA in other foods.

Compounds that are poorly soluble in water but soluble in dilute acid (e.g., ferrous fumarate, ferrous gluconate, ferrous saccharate) offer the advantages that they cause less organoleptic changes and may be selected to have bioavailabilities that are comparable to that of ferrous sulfate. At present, ferrous fumarate is widely used to fortify infant cereals, and ferrous saccharate is added to chocolate drink powders. Ferrous bisglycinate, a more expensive alternative to the other ferrous salts, has exhibited equivocal iron bioavailability, and has a tendency to cause color reactions and catalyze fat oxidation.

Water-insoluble compounds that are poorly soluble in dilute acid are the least well absorbed of the iron fortificants. In general, this class of insoluble iron fortificants comprises ferric iron in a form which precipitates from aqueous solutions having a pH above 3.5 (e.g., ferric phosphate, ferric pyrophosphate) or fine particles of elemental iron (e.g., colloidal iron). In general, fortificants in this class offer the significant advantages that they have no distinctive taste and have lower tendencies to promote fat oxidation, but special strategies may be needed to enhance bioavailability to useful ranges.

Finally, protoporphyrin-bound iron (heme-Fe) has been studied both as a dietary supplement and an additive in cereals for infants and children. Heme-Fe offers the advantages that uptake is high and predictable, but its intense color and concerns about contamination during its collection from bovine blood, together with technical difficulties in processing, residual contamination removal, and storage, deter broad use.

Physiological Uptake

In humans, iron absorption, both as heme and non-heme iron, is thought to occur predominantly in the proximal small intestine. Recent studies, however, suggest that in some populations and dietary patterns, the lower intestine may also absorb 10-15% of the iron that is ingested. In the small intestine, the efficiency of iron absorption is normally regulated in accord with iron status. In iron-replete individuals, both heme and non-heme iron absorption are down-regulated, whereas iron depletion results in enhanced iron absorption. In relative terms, non-heme iron absorption is most influenced by the iron status of the host. In iron deficiency, the amount of iron absorbed from non-heme iron sources can exceed that absorbed from heme iron.

Given iron bioavailability, the vectorial passage of iron through enterocytes of the intestine entails three phases: (1) transport of the metal across the apical membrane, (2) intracellular translocation to the basolateral surface or to stores within the cell, and (3) release of iron across the basolateral membrane into the circulation. Entry of non-heme iron into the enterocyte across the apical membrane is probably mediated by a divalent metal transporter protein, DMT-1 (also called Nramp2 or DCT-1), which is located at the apical surface of the cells. DMT-1 is a transmembrane protein that mediates cell uptake of a broad range of divalent metal cations, including Fe²⁺, Cd²⁺, Co²⁺, Zn²⁺, Ca²⁺, and so forth. In addition, uptake of heme-bound iron is mediated by specific receptors on enterocytes. In the large intestine, small (1 micron or less) particle size and/or bacterial actions may promote uptake by these and other mechanisms. In general, a total of about 1-3 mg/day of iron is absorbed from the gastrointestinal tract to maintain physiological iron homeostasis.

Enhancing Iron Absorption

As a consequence of its widespread use, both in foods and in dietary supplements, ferrous sulfate is currently the standard against which the bioavailabilities of other iron sources are compared. Among the conventional strategies that have been used to enhance the availabilities of other iron sources are:
Particle size reduction: Micronization significantly decreases particle size and increases surface area. Both factors may enhance uptake in the intestine, through more rapid solubilization and other mechanisms.
Addition of ascorbate: For over 50 years, it has been recognized that ascorbate significantly enhances iron absorption. The primary activity of ascorbate is believed to be reduction of iron from its ferric to its ferrous oxidation state, since intestinal absorption of ferrous iron is favored. Ascorbate may also enhance iron availability by preserving its solubility through metal chelation for uptake via the divalent metal transporter DMT-1 and/or through transport of the chelate via the ascorbate-transporter. Ascorbate has no effect on the bioavailability of heme-bound Fe.
Addition of organic acids: In 1947, Groen reported that non-chelated lactic, citric, malic and tartaric acids effected increases in iron absorption and non-chelated oxalic acid significantly reduced uptake in a rat model of iron availability.
Addition of amino acids: The effects of amino acids have been studied in humans and in rat models. In both humans and rodents, cysteine enhanced iron absorption. Further, in vitro studies in CaCo-2 monolayers have shown that both cysteine and (reduced) cysteinyl glycine enhanced iron uptake. In rats, histidine, ornithine, and lysine also enhanced iron uptake, whereas methionine, glutamic acid, glutamine, glycine and norleucine had no effect. A significant benefit of cysteine and related thiols over ascorbate is that the former increased iron solubility at the pH of the intestinal lumen, whereas ascorbate must be combined with iron at pH 2 to reduce and solubilize the metal.
Encapsulation in lipophilic materials: Application of a surface coating serves the dual purposes of masking adverse sensory changes that are associated with the un-encapsulated form and modifying uptake of the encapsulated material. Encapsulation may also prevent degradative interactions between the encapsulated material and its environment during long-term storage. Typical coating materials include hydrogenated oils, maltodextrins, modified cellulosics, and pH-responsive coatings (e.g., Eudragit). This strategy for enhancement of iron availability has been employed both to provide iron in dried infant formula and dried infant cereals and in dietary supplements.
Combinations of these approaches: To date, the most widely studied of the combination approaches is one in which a micronized iron source has been encapsulated (e.g., Taiyo SunActive® Fe, an iron supplement available from Taiyo International Food Company).

Iron Toxicity. In about 10-25% of the individuals who ingest iron fortificant-containing supplements in clinically relevant doses, the iron causes nausea, gastric irritation, corrosive damage to the endothelial mucosa of the intestine, and gastrointestinal injury, sometimes sufficiently severe to require hospitalization.

Symptoms of iron poisoning (e.g., nausea) occur from iron overload caused by acute ingestion of as little as 25 mg of iron/kg body weight/day. Clinically significant iron poisoning occurs at iron doses of 60 mg iron/kg body weight/day.

Once ingested, iron is absorbed in the ferrous form and subsequently oxidized to the ferric state, where it is bound to transferrin. High levels of iron compounds have a direct corrosive action on the mucosa of the intestine, which, within minutes, leads to nausea, diarrhea, and gastrointestinal hemorrhage. Depending on the dose and the iron salt, the clinical symptoms may appear to resolve, or shock, coma, and death may rapidly follow. If the dose is not sufficiently high to cause immediate death, temporary resolution is observed, although gastrointestinal obstruction and extensive, severe liver damage develop within 3-6 weeks. Within 24 hours after ingestion of toxic doses, multiorgan failure ensues with cerebral dysfunction and coma, myocardial depression, ischemic bowel, and renal and hepatic failure.

An analysis of the preceding discussion leads to the conclusion that the ideal iron fortificant/supplement will provide iron in a pharmaceutically acceptable form which can be administered to a subject by ingestion in order to safely and efficaciously deliver a nutritionally relevant amount of iron to the subject. In view of the toxicities (e.g., accidental poisoning) and dosage regimen compliance issues (e.g., failure to consistently ingest iron fortificants, owing to unpleasant side effects, unpleasant taste or odor, inconvenient tablet size, or some combination of these) which exist with regard to prior art iron fortificants/supplements, a particular need remains for an iron fortificant/supplement having a reduced risk of accidental poisoning, reduced side effects, and greater subject acceptance, which can lead to significantly improved compliance with a dosing regimen. The present invention satisfies these unmet needs.

### SUMMARY OF THE INVENTION

The present invention relates to a nutritional supplement intended for administration to a human or an animal. The supplement contains a pharmaceutically acceptable, water-soluble ferric iron (Fe³⁺) chelate capable of being reduced to the corresponding ferrous iron (Fe²⁺) chelate in response to changes in its chemical or biological environment. The supplement can also contain one or more vitamins, one or more non-ferrous minerals, or some combinations of these.

The present invention provides an oral dosage vehicle containing a ferric pyrophosphate chelate, wherein said ferric pyrophosphate chelate is chelated with citrate in a ratio sufficient to render the chelate water-soluble, a pharmaceutically acceptable excipient, and optionally, vitamins and non-mineral nutrients such as folic acid, vitamins A, B2, B6, C, D3, and niacin or nicacinamide.

Iron in the supplement can be provided in the form of a water-soluble iron chelate known as “soluble ferric pyrophosphate” or “soluble ferric pyrophosphate citrate chelate.” Soluble ferric pyrophosphate is a ferric iron chelate in which iron is bound to pyrophosphate, citrate, and phosphate in a manner that surrounds the metal ion by at least four ligands, with sufficient citrate ligands bound thereto to render the chelate very soluble in water and aqueous solutions. Soluble ferric pyrophosphate is commercially available from Dr. Paul Lohmann GmbH, Emmerthal, Germany. Soluble ferric pyrophosphate citrate chelate is a pharmaceutically acceptable ferric iron chelate in which iron is bound to pyrophosphate, citrate, and sulfate in a manner that surrounds the metal ion by at least four ligands, with sufficient citrate ligands bound thereto to render the chelate very soluble in water and aqueous solutions. Soluble ferric pyrophosphate citrate chelate is available from Rockwell Medical Technologies, Inc., Wixom, Mich. Each ferric iron chelate can be reduced to the corresponding ferrous iron chelate in response to changes in its chemical or biological environment.

Preferably, a daily dose of the iron supplement of the present invention contains at least a nutritionally relevant amount of iron, such as the Institute of Medicine's Dietary Reference Intake (DRI) of iron for an individual for whom the dose is intended. However, a clinician can order a dose comprising a greater amount of iron for ingestion under medical supervision. By way of example, the dose can provide 1-100 milligrams of iron. The dose can be provided, by way of example, as one, two, or more tablets, capsules, lozenges, or rapidly dissolving films, or another pharmaceutically acceptable oral dosage form.

Other features, advantages, and embodiments of the invention will be apparent to those of ordinary skill in the art from the following description, examples, and appended claims.

### DESCRIPTION OF THE DRAWINGS

FIG. 1 is a cartoon of a water-soluble ferric iron chelate of the present invention showing a ferric iron “core” embedded within a sphere created by surrounding ligands of citrate and pyrophosphate. The upper portion of the sphere is cut away to expose the ferric iron core.

FIG. 1A is a figure showing bioavailability of SFP chelates. Bars with no letters in common are significantly different (One-way ANOVA, p<0.05). Values are mean+SEM, n=4.

FIG. 1B is a figure showing ferritin formation from SFP-treated cells in comparison with that from FeCl₃-treated ones. The first eight bars are the same as the ones in FIG. 1A, and the significant differences among them are omitted for the clarity of the graph. Values are mean+SEM, n=4 (FeCl₃+M: n=3).

FIG. 1C is a figure showing ferritin response from five iron sources. Bars with no letters in common are significantly different. Values are mean+SEM, n=4. SFP1 was chosen to represent an average level of SFP1-4.

FIG. 2A is a figure showing bioavailability of SFP chelates. This experiment was done on 24-well plates whereas the first experiment was done on 6-well plates. As a result, the absolute values of the ferritin response were different; but the trend of the ferritin formation in the absence and presence of iron and AA was the same as seen in FIG. 1A.

FIG. 2B is a figure showing ferritin formation from four iron sources with or without AA. SFP1 was chosen to represent the average ferritin formation in response to SFP1-4 treatments. Bars with no letters in common are significantly different. Values are mean+SEM, n=3. Adding AA significantly enhanced ferritin formation from all iron sources, but the enhancing effect was especially high with SFP chelates. This was also seen in Experiment 1, FIG. 1B.

FIG. 2C is a figure showing ferritin formation from five iron sources. SFP chelates and NaFeEDTA-treated cells had significantly higher ferritin formation than the Ferrochel-, FeC₃—, and FeSO₄-treated cells. Similar trend was observed in FIG. 1C.

FIG. 3A is a figure showing the effect of in vitro digestion on iron bioavailability.

FIG. 3B is a figure showing the effect of rice and M on the bioavailability of SFP chelates. Values are mean+SEM, n=3. Bars with no letters in common are significantly different.

FIG. 3C is a figure showing the comparison of Ferritin formation from NaFeEDTA and SFP chelates, in the presence of both rice and AA. The inhibitory effect of rice was more pronounced in SFP1- and SFP2-treated cells. Values are mean+SEM, n=3.

### DETAILED DESCRIPTION OF THE INVENTION

The present invention relates to a nutritional supplement, and particularly, to an oral nutritional supplement which contains an iron fortificant comprising a ferric pyrophosphate chelate. The nutritional supplement can also include vitamins, non-ferrous minerals, and other pharmaceutically acceptable ingredients. The composition is useful for supplementing physiological iron levels by uptake of iron from the gastrointestinal tract.

The claimed iron supplement provides an improved release profile for iron, since it provides a measurable increase in iron or hemoglobin levels in blood. The invention also provides an iron supplement having reduced side effects typically associated with iron supplements comprising similar amounts of elemental or ionized iron. The dosage form can include one or more pharmaceutically acceptable excipients, flavorants, sweeteners, or some combination of these.

In one aspect, the nutritional supplement is a bioavailable iron supplement comprising an oral dosage vehicle comprising:
(a) a water-soluble ferric pyrophosphate chelate; and
(b) one or more pharmaceutically acceptable excipients;
   wherein, the dosage vehicle provides a physiological delivery of iron in the absence of a release rate modifier. The supplement can further comprise:
(c) one or more vitamins;
(d) one or more non-iron minerals;
(e) one or more flavorants;
(f) one or more sweeteners; and/or
(g) one or more release rate modifiers.

The oral dosage vehicle of the nutritional supplement can be a pharmaceutically acceptable tablet, capsule, caplet, granule, particulate, agglomerate, spansule, chewable tablet, lozenge, troche, solution, suspension, rapidly dissolving film, elixir, gel, or syrup. Dosage vehicles which persist in the mouth (e.g., lozenges and troches) are not preferred, given the unpleasant taste associated with some nutrients (e.g., B vitamins). In a preferred form, the active ingredients of the iron supplement are mixed with the one or more excipients and compressed to form a tablet. The tablet is then optionally coated with one or more coats, at least one of which preferably comprises a flavorant.

In one embodiment, the nutritional supplement is a pharmaceutically acceptable oral dosage vehicle comprising
(a) from about 0.1 milligram to about 2.0 milligrams, preferably about 1.0 milligram, of folic acid, or a pharmaceutically acceptable salt form thereof;
(b) from about 100 I.U. to about 4000 I.U., preferably about 100-2000 I.U. (e.g., 1000 I.U.), of beta-carotene or another form or precursor of vitamin A (e.g., vitamin A acetate);
(c) from about 0.2 milligram to about 8 milligrams, preferably about 2 milligrams, of Vitamin B1;
(d) from about 0.5 milligram to about 10 milligrams, preferably about 3 milligrams, of Vitamin B2;
(e) from about 2 milligrams to about 20 milligrams, preferably about 10 milligrams, of Vitamin B6;
(f) from about 2 micrograms to about 20 micrograms, preferably about 12 micrograms, of Vitamin B12;
(g) from about 20 milligrams to about 200 milligrams, preferably about 120 milligrams, of Vitamin C dosed in the form of ascorbic acid and/or a pharmaceutically acceptable salt thereof (e.g., sodium ascorbate);
(h) from about 5 milligrams to about 40 milligrams, preferably about 20 milligrams, of niacin or niacinamide;
(i) from about 1 milligram to about 100 milligrams of iron provided as a water-soluble ferric iron chelate selected from the group consisting of soluble ferric pyrophosphate and soluble ferric pyrophosphate citrate chelate; and
(j) one or more pharmaceutically acceptable excipients; wherein the solid dosage provides a controlled release of the iron absent a release rate modifier.

The iron supplement solid dosage vehicle can further comprise a release rate modifier that modulates the delivery of an iron compound, vitamin, mineral or other active ingredient.

The invention includes a method of alleviating an iron deficiency related disease or disorder in an animal. This method comprises administering an iron-containing nutritional supplement described herein to the animal (e.g., prior to, during, or following onset of the disease or disorder). Examples of diseases and disorders which can be alleviated using this method include anemia, birth defects, low birth weight, and anemia of chronic disease. The animal to which the supplement is administered is preferably a human, and can be one who is afflicted with the disease or disorder, or who is at risk for developing the disease or disorder. By way of example, the nutritional supplement can be administered to a pregnant or lactating woman, or to a woman who anticipates becoming pregnant. The nutritional supplement can also be administered to a woman who is nursing an infant for the purpose of providing the nutrients in the supplement to the infant. By way of further example, the nutritional supplement can be administered to a human of either gender and any age who suffers from anemia of chronic disease.

The invention relates, in one aspect, to the discovery that nutritional supplements which exhibit advantageous properties, relative to prior art nutritional supplements, can be made by providing iron as a water-soluble ferric iron chelate. Such supplements can, and preferably do, contain one or more vitamins and non-ferrous minerals.

The compositions and methods described herein are useful for providing iron to animals, and are intended to be used, for example, to administer iron to men and women, including individuals afflicted with anemia of chronic disease, pregnant women, women anticipating pregnancy, and lactating women. The compositions and methods can also be used to administer iron together with one or more vitamins or non-ferrous minerals to men, women, children or infants. By way of example, the compositions described herein include prenatal vitamin supplements containing iron, folic acid, and optionally, other vitamins and minerals. Further by way of example, the compositions include daily vitamin/mineral supplements for administration to animals, regardless of age, gender and species.

The particular combination of iron, vitamins, minerals, and other ingredients in the claimed iron-containing nutritional supplement advantageously provides a product with high nutritional value, high bioavailability, and reduced side effects, relative to prior art nutritional supplements, particularly with respect to those which contain a ferrous iron compound. The iron supplement of the invention provides a measurable improvement over other known iron supplements in terms of iron release profile and a reduction in the severity or number of side effects, which are typically associated with administration of iron to animals.

For example, when a composition described herein is used as a prenatal daily multi-vitamin/mineral supplement, the composition preferably comprises amounts of vitamins and minerals in the following ranges:
(a) about 1-100 milligrams of iron (preferably at least about 15, 30, 45, 60, or 90 milligrams);
(b) about 0.1-2.0 milligrams of folic acid (preferably at least about 1-1.2 milligrams);
(c) about 100-2000 International Units (I.U.) of vitamin A or a substitute for vitamin A (preferably at least about 1000-1100 I.U.);
(d) about 0.2-8 milligrams of vitamin B1 (preferably at least about 2-2.4 milligrams);
(e) about 0.5-10 milligrams of vitamin B2 (preferably at least about 3-3.45 milligrams);
(f) about 2-50 milligrams of vitamin B6 (preferably at least about 10-12 milligrams);
(g) about 2-20 micrograms of vitamin B12 (preferably at least about 12-14.4 milligrams);
(h) about 20-200 milligrams of vitamin C (preferably at least about 120-132 milligrams);
(i) about 100-800 I.U. of vitamin D3 (preferably at least about 400-440 I.U.); and
(k) about 5-40 milligrams of one of niacin and niacinamide (preferably at least about 20-22 milligrams of niacinamide or an equivalent molar amount of niacin).

According to the method of the present invention, an iron fortificant of the present invention is administered, alone or in combination with other substances (e.g., along with materials necessary to form a pharmaceutically acceptable oral dosage vehicle as a delivery vehicle for the iron fortificant; in a tablet or caplet; in a hard gelatin capsule; together with a binder or other pharmaceutically useful substance) in sufficient quantities to enable iron absorption from the gastrointestinal tract. The ferric iron chelate is administered orally in a pharmaceutically acceptable dosage vehicle. For convenience, the total daily dosage may be divided and administered in portions during the day if desired or at one time, morning, afternoon, night as well as biphasic, triphasic, etc. Controlled, delayed (e.g., enteric), and sustained release formulations are within the scope of the invention and, for convenience, are termed “controlled release” formulations.

The term “active ingredient” as used herein encompasses any material having physiological activity such as a vitamin, mineral, flavorant, sweetener, or other nutrient and combinations thereof.

The term “excipient material” is intended to mean any compound forming a part of the formulation which is not intended to have biological activity itself and which is added to a formulation to provide specific characteristics to the dosage form, including by way of example, providing protection to the active ingredient from chemical degradation, facilitating release of a tablet or caplet from equipment in which it is formed, and so forth.

By the terms “treating” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein encompasses any treatment of a disease in an animal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms or conditions.

The phrase “therapeutically effective” is intended to qualify the amount of water-soluble ferric iron chelate for use in the orally administered therapy which will achieve the goal of returning the iron or hemoglobin levels to more normal clinical values by providing iron that is available for absorption in the gastrointestinal tract, while avoiding adverse side effects typically associated with iron supplements or iron fortificants.

Included within the scope of this invention is a method of treating iron deficiency or iron deficiency anemia in a warm-blooded animal using pharmaceutical compositions comprising a water-soluble ferric iron chelate and a suitable pharmaceutical carrier.

For the purpose of this disclosure, a warm-blooded animal is a member of the animal kingdom which includes but is not limited to mammals and birds. The most preferred animal of this invention is human.

Over the past decade, the U.S. Food and Drug Administration have barred the use of a broad spectrum of materials that are purported to have therapeutic benefit on the basis of historical use or anecdotes. The bars have followed evaluation of a material using tests and assays that are validated, current, state of the art methods, where the testing showed that the material did not have the purity, quality, bioavailability, or therapeutic benefit that was claimed.

Surprisingly, the inventor has discovered that, compared to conventional iron fortificants, a water-soluble ferric iron chelate of the present invention provides unexpectedly high iron bioavailability following ingestion, a biological and physiological action having distinct advantages to a subject requiring treatment for iron deficiency or anemia. While not wishing to be bound by any particular hypothesis or theory, the inventor believes that three factors support using a ferric iron chelate of the present invention as an iron fortificant/supplement: (1) Its ability, without decomposition, to be reduced from a ferric iron chelate to a ferrous iron chelate and re-oxidized from a ferrous iron chelate to a ferric iron chelate in response to changes in its chemical or biological environment. (2) Its ability to provide iron to a subject by absorption from the gastrointestinal tract of the subject. (3) Safety. These factors are discussed in greater detail below.

Ability to be reduced from a ferric iron chelate to a ferrous iron chelate and re-oxidized from a ferrous iron chelate to a ferric iron chelate in response to changes in its chemical or biological environment without undergoing decomposition. The pH of the gastrointestinal tract changes from a pH of 1-2 in the stomach to a pH of 6 to 8 in the proximal small intestine and colon, respectively. The inventor has unexpectedly discovered that each ferric iron chelate of the invention is stable in solutions having a pH of greater than about 3. Thus, each chelate is stable in the stomach. In an environment having a pH of 6 to about 8, the chelate is reduced to a ferrous iron chelate that is also stable and water-soluble at the higher pH. Although the chelate does not decompose in either environment, ferrous iron from the chelate is highly bioavailable for uptake from the gastrointestinal tract.

Ability to Provide Iron to a Subject by Absorption from the Gastrointestinal Tract of the Subject.

In Example 1, the bioavailability of a water-soluble ferric iron chelate of the present invention is compared to that of several conventional iron fortificants. The test system was a validated method using Caco-2 cells, which take up iron from the culture medium and process it into ferritin. It is known that iron which is available to Caco-2 cells is comparably bioavailable to animals for uptake from the gastrointestinal tract. The results of testing using this system showed that a water-soluble ferric iron chelate of the present invention is more bioavailable than conventional iron fortificants. Further, the inventor unexpectedly discovered that bioavailability of a water-soluble ferric iron chelate of the present invention is significantly enhanced by provision of ascorbate, both in the absence and presence of foods that would otherwise inhibit uptake of iron from the gastrointestinal tract.

A priori, because SFP is a citrate-containing chelate and because non-chelated citrate is an iron uptake enhancer, one might have predicted that added ascorbate would have little effect. Thus, the significant enhancement in iron uptake and ferritin production that was observed when Caco-2 cells were exposed to both SFP and ascorbate was a surprising discovery by the inventor.

Safety. Iron toxicity is related to the direct exposure of and close contact between ionized iron and cells and tissues. In general, because of its excellent solubility, ferrous iron is more toxic than ferric iron. However, it is also known that ionized ferric iron can be reduced to ferrous iron. Ionized iron that is surrounded by ligands in at least a 1:2 ratio of metal to ligands restricts unwanted reactions with dietary components, neutralizes the valence of the iron, and protects the cell and tissue surfaces of the gastrointestinal tract from being irritated by close contact with the iron atom. Provided that the chelating bonds are sufficiently strong to resist cleavage by digestion or through reactive natural foodstuffs, a chelate can protect ionized iron sufficiently long to be absorbed and utilized nutritionally. There is precedence in the natural iron source from animals known as heme for demonstrating this protection gained from chelates. Surprisingly, Applicant observed that a ferric iron chelate of the present invention, comprising a ferric iron core surrounded by citrate and pyrophosphate ligands, also provides this protection against iron-related toxicity. Thus, neither Caco-2 cells (Example 1) nor humans experienced irritation or toxicity after exposure to therapeutically beneficial concentrations of the water-soluble ferric iron chelate.

DOSAGE FORMS. The pharmaceutical compositions of this invention can be administered by any means that effects contact of the therapeutically active ingredients (i.e., active ingredients) with the site of action in the body of a warm-blooded animal. A most preferred administration is by the oral route (i.e., ingestion). The active ingredients can be administered by the oral route in solid dosage forms, such as tablets, capsules, and powders, or in liquid dosage forms, such as elixirs, syrups, and suspensions. The pharmaceutical composition is preferably made in the form of a dosage unit containing a particular amount of each active ingredient.

In general, the pharmaceutical compositions of this invention can be prepared by conventional techniques, as are described in Remington's Pharmaceutical Sciences, a standard reference in this field [Gennaro A R, Ed. Remington: The Science and Practice of Pharmacy. 20^{th} Edition. Baltimore: Lippincott, Williams & Williams, 2000]. For therapeutic purposes, the active components of this invention are ordinarily combined with one or more excipients appropriate to the indicated route of administration. Such capsules or tablets may contain a controlled-release formulation as may be provided in a dispersion of active compound in hydroxypropyl methylcellulose or related material known to alter the kinetics of release of the active agent. Solid dosage forms can be manufactured as sustained release products to provide for continuous release of medication over a period of hours using known pharmaceutical techniques. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance.

The following examples present hypothetically useful therapeutic applications of representative pharmaceutical compositions of the present invention and their anticipated outcomes in treating iron deficiency in subjects requiring such treatment. The examples are representative of the scope of the invention, and as such are not to be considered or construed as limiting the invention recited in the appended claims.

### EXAMPLE 1

### Bioavailability of Iron Supplements Using the Caco-2 Cell Model

We conducted three in vitro experiments using the Caco-2 cell model to address the following questions:
1. Compare the bioavailability of four Soluble Ferric Pyrophosphate (SFP) chelates in solution to that of conventional iron sources such as FeSO₄, ferrous bisglycinate (Ferrochel®), FeCl₃, and NaFeEDTA.
2. Assess the bioavailability of SFP chelates and other iron compounds in the absence or presence of rice (an iron-uptake inhibitor) and/or ascorbic acid (an iron-uptake promoter), using in vitro digestion.

Materials and Methods

Materials. Four test samples of Soluble Ferric Pyrophosphate (SFP; a ferric iron chelate) were characterized analytically and provided for Caco-2 cell study. The samples comprised:

| | |
|---|---|
| SFP-1 - Lot: BLS512305-SFPTC | Iron content: 8.6% by weight (w/w) |
| SFP-2 - Lot: BLS512426-SFPTC | Iron content: 8.6% w/w |
| SFP-3 - Lot: 126412 | Iron content: 11.7% w/w |
| SFP-4 - Lot: BLS511913-SFPDC | Iron content: 10.2% w/w |

Cell culture. Caco-2 cells were obtained from the American Type Culture Collection at passage 17 and used in experiments at passage 29-35. Cells were seeded at a density of 50,000 cells/cm² in collagen-treated 6-well or 24-well plates. The integrity of the monolayer was verified by optical microscopy. The cells were cultured at 37° C. in an incubator with a 5% CO₂ and 95% air atmosphere at constant humidity. The cells were maintained in Dulbecco's Modified Eagle Medium (DMEM) plus 1% antibiotic/antimycotic solution, 25 mmol/L HEPES and 10% fetal bovine serum; the medium was changed every 2 days. Two days before the experiment, the growth medium was removed from each culture well, and the cell layer was washed and maintained with Minimum Essential Media (MEM) at pH 7.0. The MEM was supplemented with 10 mmol/L PIPES, 1% antibiotic/antimycotic solution, 4 mg/L hydrocortisone, 5 mg/L insulin, 5 μg/L selenium, 34 μg/L triiodothyronine and 20 μg/L epidermal growth factor. This enriched MEM contained less than 80 μg iron/L. Iron uptake experiments were conducted 13 days post-seeding.

Harvesting of Caco-2 cells for ferritin analysis. Growth medium was first removed from the culture well by aspiration, and the cells were washed twice with a solution containing 140 mmol/L NaCl, 5 mmol/L KCl and 10 mmol/L PIPES at pH 7.0. The cells were harvested by adding an aliquot of de-ionized water and placing in a sonicator at 4° C. for 15 min. Cells were frozen at −20° C. immediately after harvest until later analysis. Ferritin concentration and total protein concentration were determined on an aliquot of the harvested cell suspension with a one-stage sandwich immunoradiometric assay and a colorimetric assay, respectively. Caco-2 cells synthesize ferritin in response to increases in intracellular iron concentration. Therefore, the ratio of ferritin/total protein expressed as ng ferritin/mg protein, was used as an index of the cellular iron uptake.

In-solution treatment. Caco-2 cells were incubated in MEM mixed with aqueous solutions of desired iron source, in the absence or presence of ascorbic acid (M). The final concentration of all iron compounds was 20 μmol/L. When added, the final AA concentration was 400 μmol/L. Treated cells were incubated with cells for 20 hours and then harvested. Cellular ferritin and total protein was then analyzed and compared among all treatments.

In vitro digestion. The pH of each sample was adjusted to pH 2.0 with 5.0 mol/L HCl. An aliquot of pepsin solution was added at the concentration of 0.5 mL pepsin solution/10 mL sample. This mixture was placed on a rocking shaker for an hour (55 oscillations/min). The pH of the sample was raised to pH 6.0 with 1 mol/L NaHCO₃ before the addition of pancreatin-bile extract (2.5 mL extract/10 mL sample). The pH was then adjusted to pH 7.0 with NaOH solution, and the volume was brought to 15 mL with 120 mmol/L NaCl and 5 mmol/L KCl. The growth medium was removed from each cell well before a fresh 1 mL aliquot of MEM was added to each cell well. A sterilized insert ring, fitted with a dialysis membrane, was then inserted into the well, thus creating the two-chamber system. A 1.5-mL aliquot of the intestinal digest was pipetted into the upper chamber. The plate was incubated for 120 minutes with 6 oscillations/min rocking speed. When the intestinal digestion was terminated, the insert ring and digest were removed. The solution in the bottom chamber was allowed to remain on the cell monolayer and an additional 1 mL of MEM was added to each well. The cell culture plate was then returned to the incubator for an additional 20 hours, after which the cells were harvest for analysis.

### EXPERIMENT 1

### In-Solution Treatment, Test for Bioavailability

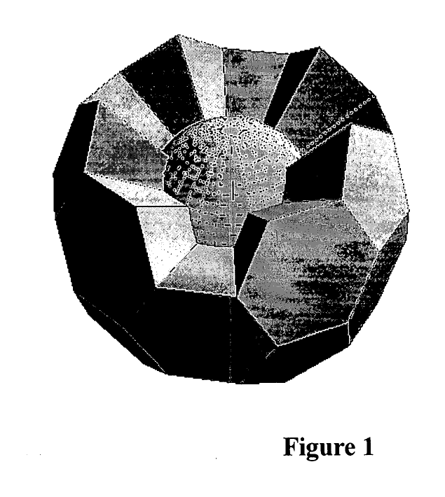

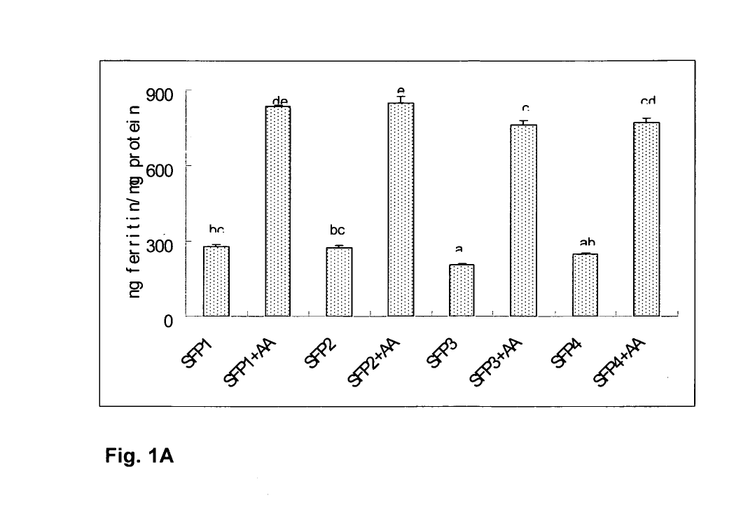

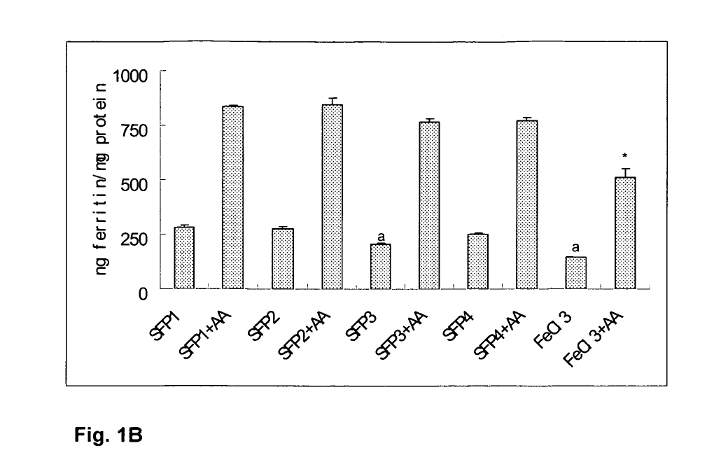

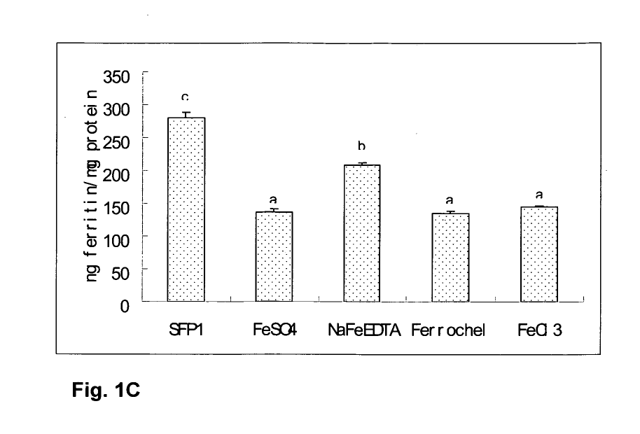

This experiment addressed the first of the Specific Aims of the study and answered Technical Questions 1 and 2 by comparing the availability of SFP, a ferric iron chelate, to that of conventional Fe compounds such as ferrous sulfate, NaFeEDTA, ferrous bisglycinate (Ferrochel), and ferric chloride, and by determining the effects of an iron absorption enhancer, ascorbic acid, on iron bioavailability. The final concentration of all iron compounds was 20 μmol/L. When added, the final AA concentration was 400 μmol/L. Six-well plates were used. Each sample was prepared in quadruplicate, and the results were averaged. Experimental data are presented graphically in FIGS. 1A through 1C.

As FIG. 1A shows, the four SFP chelates induced similar ferritin formation in Caco-2 cells. Adding AA further enhanced ferritin formation significantly.

As FIG. 1B shows, in the absence of AA, FeCl₃— and SFP-3-treated cells had similar ferritin formation. In the presence of AA, ferritin formation in FeCl₃-treated cells was significantly lower than that in SFP-treated ones (indicated by *).

As FIG. 1C shows, in the absence of enhancers, inhibitors and food components, the bioavailability of SFP chelates were significantly higher than that of NaFeEDTA, FeSO₄, Ferrochel and FeCl₃.

### EXPERIMENT 2

### In-Solution Treatment, Test for Bioavailability and Reproducibility

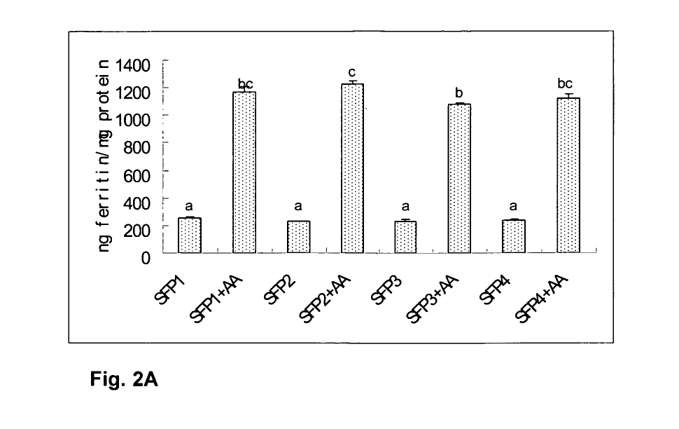

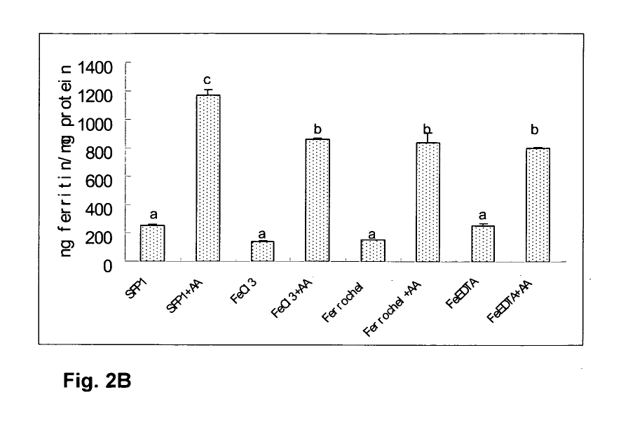

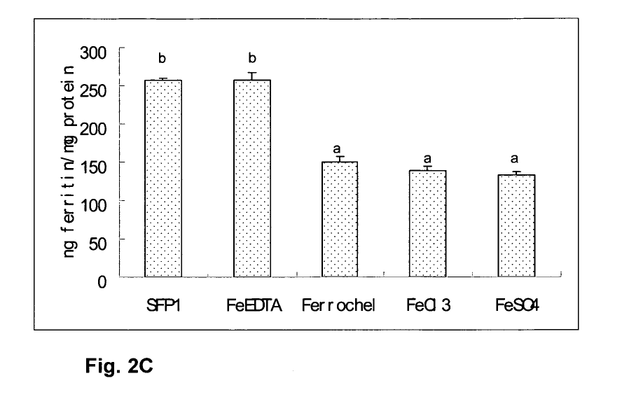

This experiment compared the availability of SFP, a ferric iron chelate, to that of conventional Fe compounds such as ferrous sulfate, NaFeEDTA, ferrous bisglycinate (Ferrochel), and ferric chloride. The final concentration of all iron compounds was 20 μmol/L. When added, the final AA concentration was 400 μmol/L. Twenty-four well plates were used. (The results, when compared with those of Experiment 1, verify the reproducibility of the data.) Each sample was prepared in triplicate, and the results were averaged. Experimental data are presented graphically in FIGS. 2A through 2C.

As the data in FIG. 2A show, the trend of the ferritin formation in the absence and presence of iron is the same in Experiments 1 and 2.

As the data in FIG. 2B show, adding AA significantly enhanced ferritin formation from all iron sources, but the enhancing effect was especially high with SFP chelates. This replicates the trend that was seen in Experiment 1, FIG. 1B.

As FIG. 2C shows, ferritin formation from SFP chelates and NaFeEDTA treated cells was significantly higher than ferritin formation from Ferrochel, ferrous sulfate, or ferric chloride treated cells. These data replicate the trend observed in Experiment 1, FIG. 1C.

### EXPERIMENT 3

### In Vitro Digestion, Test for the Effect of Food on Bioavailability

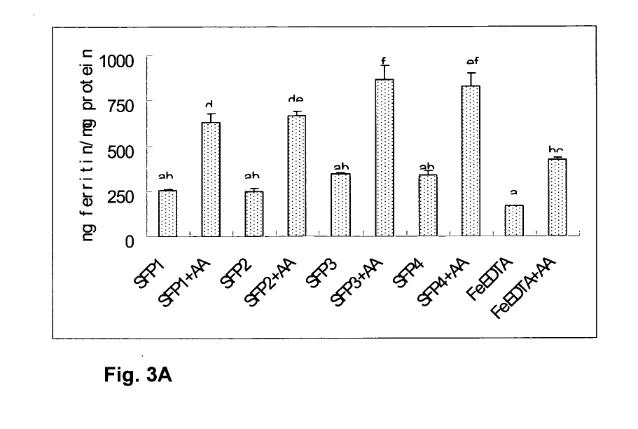

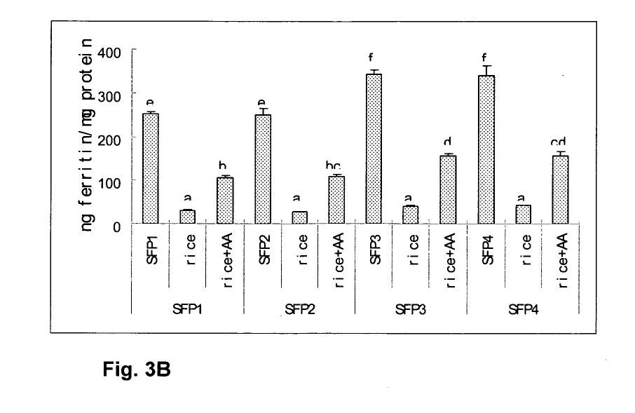

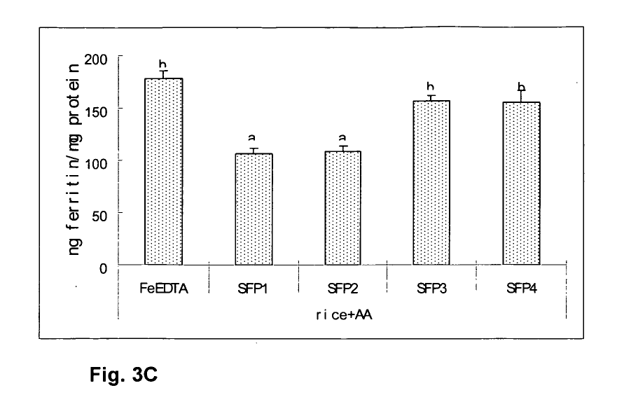

This experiment provided data showing the effects of inhibitors on iron bioavailability. In vitro digestion was used to mimic digestion in the gastrointestinal tract, and rice, a known iron uptake inhibitor, was added to mimic the effects of food on iron bioavailability. The experiment was conducted in 6-well plates. Rice was added as Nshiki Rice reference, which had been cooked, freeze-dried, and then ground into fine powder. By assay, rice contained 2.7 ppm Fe and 2.86 μmol phytate/gram rice. During in vitro digestion, 1 g rice was added to each treatment prior to pepsin digestion. The final concentration of iron (in the upper chamber) was 50 μmol/L, and if present, the final concentration of Ascorbic acid (AA, in the upper chamber) was 1000 μmol/L. All treatments were done in triplicate. Data are summarized graphically in FIGS. 3A through 3C.

Two general conclusions regarding iron bioavailability are drawn from the data in FIG. 3A. First, a comparison of these data with the data from experiments 1 and 2 confirms that in vitro digestion did not affect the bioavailability of the iron sources very much. Secondly, adding AA, an iron absorption enhancer, enhanced ferritin formation from SFP-treated cells more than it did to NaFeEDTA-treated ones.

As shown in FIG. 3B, adding rice, an iron absorption inhibitor, significantly decreased iron bioavailability from all SFP iron sources. Adding AA in the presence of rice enhanced ferritin formation significantly, but the ferritin values are still much lower than those from cells treated without rice.

The data in FIG. 3C compare the effects of rice and ascorbic acid on the bioavailability of iron in NaFeEDTA and each of the four SFP chelates. These data indicate that iron bioavailability from NaFeEDTA, SFP-3 and SFP-4 was the same but that significantly less iron was available from SFP-1 and SFP-2.

### EXAMPLE 2

### Efficacy of a Ferric Iron Chelate of the Present Invention in Subjects with Iron Deficiency Anemia

Thirty subjects will be recruited from blood donors who meet the following criteria: (1) menstruating non-pregnant women between the ages of 18 and 40 years and (2) deferral for repeat blood donation because of hematocrit <38%. Twice each day for 12 weeks they will ingest a gelatin capsule containing 12.5 mg iron. Blood samples will be drawn at 0, 1, 3, 6, 9, and 12 weeks for determination of free erythrocyte protoporphyrin (FEP), serum ferritin, serum iron, TIBC, percent saturation of TIBC, and complete blood count (CBC) including hemoglobin, mean cellular volume (MCV), white blood cells (WBC), and platelets. In addition, serum bilirubin, SGOT, SGPT, alkaline phosphatases, and creatinine will be measured at 0, 1, 3, and 12 weeks. Side effects will be recorded on standard forms that include space to record constipation, diarrhea, heartburn, nausea, abdominal cramps, headache, weakness, and “unpleasant taste” at weeks 1, 3, 6, 9, and 12.

An estimate for the absorption of iron will be made by calculating the increase in hemoglobin iron (hematocrit) and storage iron (serum ferritin) between weeks 0 and 12 of the study and using the following equation:

Total amount of iron absorbed=increase in hemoglobin iron+increase in storage iron

The increase in hemoglobin iron will be calculated using the equation:

Increase in hemoglobin iron=(increase in hemoglobin (g/100 mL))×(3.47 mg Fe/g hemoglobin)×(assumed body weight of 60 kg)×(60 mL blood/kg body weight)

The increase in storage iron will be calculated by assuming that 1 μg/L of serum ferritin represents approximately 10 mg of storage iron if the serum ferritin is greater than 12 μg/L and that storage iron is absent if the serum ferritin is less than 12 μg/L. Since at week 0, the serum is expected to be less than 12 μg/L in an anemic individual, the equation will be the following:

Increase in storage iron (mg)=(serum ferritin (μg/L) at week 12)−(12 μg/L)×10

Expected results for the study are shown in Table 1. In addition, it is reasonable to expect that less than about 10% of the individuals in the study will complain of side effects and that none of the complaints will refer to a clinically significant side effect (e.g., a side effect that will require cessation of treatment or hospitalization). Taken together, these results will show that a ferric iron chelate of the invention will correct iron deficiency anemia in humans.

TABLE 1
Short-term Iron Chelate Therapy for Iron Deficiency Anemia

| | Normal | | | |
|---|---|---|---|---|
| | Value | Week 0 | Week 12 | p |
| Hemoglobin (g/dL) | 12.0-16.0 | 10.8 ± 2 | 12.9 ± 1 | 0.0001 |
| MCV (fL) | 81.0-99.0 | 80.2 ± 1 | 88.3 ± 1 | 0.0001 |
| FEP (μg/dL, whole | 10-35 | 48 ± 3 | 27 ± 1 | 0.0001 |
| blood) | | | | |
| Serum ferritin (μg/L) | 12-250 | 5.0 ± 0.5 | 15 ± 1 | 0.0001 |
| Serum iron (μg/dL) | 80-200 | 71 ± 10 | 82 ± 1 | 0.0001 |
| TIBC (μg/dL) | 250-435 | 417 ± 10 | 358 ± 1 | 0.0001 |
| % Saturation | 18-50 | 17 ± 2 | 23 ± 1 | 0.0001 |

### EXAMPLE 3

### Iron Solubility and Bioavailability in Milk. A. In Vitro Evaluation

The choice of iron fortificant in milk is challenging because conventional iron in milk has low bioavailability due to the presence of absorption inhibitors in milk such as casein, calcium, whey protein and phosphates. Two experiments will be completed to demonstrate the solubility and bioavailability of an iron chelate of the present invention in milk. The evaluation of the various iron fortificants in milk will be based on measurements of ferrous dialyzable, total (ferrous and ferric) dialyzable, ferrous soluble and total (ferrous and ferric) soluble iron. These indices have been employed in the literature for the prediction of iron bioavailability in the in vitro model employed herein. Of these measures, ferrous dialyzable iron has been evaluated as a preferable index because it exhibits better correlation with results on iron uptake by cells and with data on iron absorption by humans. Pasteurized milk will be fortified with ascorbic acid (5 mg ascorbic acid/100 mL sample) and an iron fortificant (1.2 mg iron/100 mL sample) under laboratory conditions. The concentration of 1.2 mg iron/100 mL was chosen because the typical concentration used in milk products directed towards older infants and toddlers is in the range 1.1-1.3 mg iron/100 mL. The concentration of 5 mg ascorbic acid/100 mL was chosen because this is the ascorbic acid concentration used in commercial milk samples.

The results are expected to demonstrate that a water-soluble ferric iron chelate of the present invention will be soluble and stable in milk and will provide physiological and bioavailable concentrations of iron in the gastrointestinal tract of a subject drinking the milk.

B. Evaluation in children. One hundred and fifty young children (at least 100 being 1-year old at the commencement of the study) will be given 3 mg iron as a ferric iron chelate of the invention in 1 L of cow's milk per day. Hemoglobin concentrations will be measured initially and at 133±13 and 222±2 days into the study. Mean and standard deviations for each sampling are expected to rise from 9±1.5 (initial) to 10.5±1.5 (Day 133) to 11.0±1.5 g hemoglobin/dL (Day 222). Observation of these increases will demonstrate the repletion of iron deficiency anemia in the children over the course of the study. The data will be additionally divided by degree of anemia, in which an initial blood hemoglobin of 9.4 g hemoglobin/dL whole blood or less is deemed the most severe and an initial blood hemoglobin of 9.5-11.0 g hemoglobin/dL is deemed less severe. Children having hemoglobin levels of 11.1 g/dL will be considered normal. Over the course of the study, it is expected that the greatest changes in hemoglobin will be noted in the most severely anemic group. Among children with normal hemoglobin values, there will be no significant differences in hemoglobin amounts at any of the measurement times (P>0.10).

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The above is a detailed description of particular embodiments of the invention. Those of ordinary skill in the art should, in light of the present disclosure, appreciate that obvious modifications of the embodiments disclosed herein can be made without departing from the spirit and scope of the invention. All of the embodiments disclosed and claimed so herein can be made and executed without undue experimentation in light of the present disclosure. The full scope of the invention is set out in the claims that follow and their equivalents.

Accordingly, the claims and specification should not be construed to unduly narrow the full scope of protection to which the present invention is entitled.

What is claimed is:

1. An oral dosage vehicle comprising:
a water-soluble ferric pyrophosphate chelate,
a pharmaceutically acceptable excipient, and
optionally, vitamins and non-metallic nutrients.

2. The oral dosage vehicle of claim 1 wherein said ferric pyrophosphate chelate is ferric pyrophosphate chelated with citrate in a ratio sufficient to render the chelate water soluble.

3. The oral dosage vehicle of claim 1 wherein said optional vitamins and non-mineral nutrients are selected from the group consisting of folic acid, vitamin A or a substitute for Vitamin A, vitamin B2, vitamin B6, vitamin B12, vitamin C, vitamin D3, and niacin or nicacinamide.

4. The oral dosage vehicle of claim 1, comprising
(a) from about 0.1 milligram to about 2.0 milligrams, preferably about 1.0 milligram, of folic acid, or a pharmaceutically acceptable salt form thereof;
(b) from about 100 I.U. to about 4000 I.U., preferably about 100-2000 I.U. (e.g., 1000 I.U.), of beta-carotene or another form or precursor of vitamin A (e.g., vitamin A acetate);
(c) from about 0.2 milligram to about 8 milligrams, preferably about 2 milligrams, of Vitamin B1;
(d) from about 0.5 milligram to about 10 milligrams, preferably about 3 milligrams, of Vitamin B2;
(e) from about 2 milligrams to about 20 milligrams, preferably about 10 milligrams, of Vitamin B6;
(f) from about 2 micrograms to about 20 micrograms, preferably about 12 micrograms, of Vitamin B12;
(g) from about 20 milligrams to about 200 milligrams, preferably about 120 milligrams, of Vitamin C dosed in the form of ascorbic acid and/or a pharmaceutically acceptable salt thereof (e.g., sodium ascorbate);
(h) from about 5 milligrams to about 40 milligrams, preferably about 20 milligrams, of niacin or niacinamide;
(i) from about 1 milligram to about 100 milligrams of iron provided as a water-soluble ferric pyrophosphate chelate selected from the group consisting of soluble ferric pyrophosphate and soluble ferric pyrophosphate citrate chelate; and
(j) one or more pharmaceutically acceptable excipients;
wherein the composition provides a controlled release of the iron absent a release rate modifier.

5. A method for supplementing nutrients in a subject having nutritional deficiencies comprising the step of administering to said subject a composition comprising 1-100 mg water-soluble ferric pyrophosphate chelate, vitamins, non-ferrous minerals, and other ingredients.

6. The method of claim 5, wherein said composition is substantially free of other added vitamins and minerals.

7. The method of claim 5, wherein said composition further comprises a pharmaceutically acceptable carrier.

8. The method of claim 5, wherein said composition is administered to an individual requiring treatment for iron deficiency.

9. An oral dosage vehicle comprising a water-soluble ferric pyrophosphate chelate, ascorbate, an excipient; and optionally, vitamins and non-metallic nutrients.
